(19) United States
(12) Patent Application Publication
(10) Pub. No.: US 2022/0400695 A1
(43) Pub. Date: Dec. 22, 2022

(54) PEANUT MILK PRODUCT WITH SUPERIOR PEANUT TASTE, AROMA AND TO IMPROVE GUT MICROBIOTA AND PREPARATION PROCESS AND USE THEREOF

(71) Applicant: AGV PRODUCTS CORP., Minsyong Township (TW)

(72) Inventors: Kwan-Han CHEN, Minsyong Township (TW); Hung-Chi HSIAO, Minsyong Township (TW); Chun-Liang CHOU, Minsyong Township (TW); Li-Chueh HUANG, Minsyong Township (TW); Yun-Wen CHEN, Minsyong Township (TW); Pei-Hsiu HUANG, Minsyong Township (TW)

(21) Appl. No.: 17/842,861

(22) Filed: Jun. 17, 2022

(30) Foreign Application Priority Data
Jun. 22, 2021 (TW) 110122732

(51) Int. Cl. A23C 9/12; A23C 21/02; C12P 19/14

(52) U.S. Cl. CPC A23C 9/1206; A23C 21/023; C12P 19/14; C12Y 302/01023

(57) ABSTRACT

Disclosed herein is a process for producing a peanut milk product, which includes subjecting a milk material to a hydrolysis reaction with lactase and transglucosidase to obtain a milk product, immersing peanut kernels in water to obtain swelled peanut kernels, subjecting the swelled peanut kernels to a steam cooking treatment to obtain steamed peanut kernels, and admixing the milk product with the steamed peanut kernels, followed by subjecting a mixture thus obtained to a heating treatment, so as to obtain the peanut milk product. A method for improving gut health using the peanut milk product and a canned food containing the peanut milk product are also disclosed.

### CROSS-REFERENCE TO RELATED APPLICATION

This application claims priority of Taiwanese Patent Application No. 110122732, filed on Jun. 22, 2021.

### FIELD

The present disclosure relates to a peanut milk product and a process of preparing the same. The present disclosure also relates to use of the peanut milk product to improve gut health.

### BACKGROUND

CN 104757624 A discloses a method for producing a peanut kernel soup, which includes: (a) preparing a sugar solution; (b) adjusting the sweetness and pH value of the sugar solution, so that the sugar solution has a sugar content of 11.5-12.5° Brix and a pH value of 6.5-7.5; (c) immersing peanut kernels in water, followed by washing with water, and subsequently precooking the washed peanut kernels at a temperature ranging from 80° C. to 100° C. for a time period ranging from 20 minutes to 30 minutes, so as to obtain precooked peanut kernels; and (d) admixing the sugar solution obtained in step (b) with the precooked peanut kernels obtained in step (c) in a bottle, followed by sealing, sterilization and cooling packaging. The sterilization in step (d) is conducted at a temperature of 121±1° C. for a time period ranging from 30 minutes to 50 minutes.

The method of CN 104757624 A improves the taste and aroma of the sugar solution by adjusting the sugar content and pH value of the sugar solution, and retains the structure of the peanut kernels during the processing, so that the peanut kernel soup has a delicate mouthfeel.

In spite of the aforesaid, there is still a need to develop a process for preparing a peanut milk product that has good peanut granule texture and flavor and can effectively improve gut health.

### SUMMARY

Therefore, in a first aspect, the present disclosure provides a process for producing a peanut milk product, which can alleviate at least one of the drawbacks of the prior art. The process includes:
(a) subjecting a milk material to a hydrolysis reaction with lactase and transglucosidase at a temperature ranging from 40° C. to 60° C. for a time period ranging from 50 minutes to 70 minutes, so as to obtain a first milk product containing galacto-oligosaccharides and isomalto-oligosaccharides, the first milk product being optionally further subjected to heating at a temperature ranging from 70° C. to 100° C. to inactivate the lactase and transglucosidase, and subsequently to dilution with water, so as to obtain a second milk product;
(b) immersing peanut kernels in water at a temperature ranging from 70° C. to 80° C. for a time period ranging from 3 hours to 4 hours, so as to obtain swelled peanut kernels;
(c) subjecting the swelled peanut kernels obtained in step (b)to a steam cooking treatment at a temperature ranging from 105° C. to 125° C. for a time period ranging from 5 minutes to 30 minutes, so as to obtain steamed peanut kernels; and
(d) admixing the first milk product or the second milk product obtained in step (a) with the steamed peanut kernels obtained in step (c), followed by subjecting a mixture thus obtained to a heating treatment at a temperature ranging from 120° C. to 130° C. for a time period ranging from 36 minutes to 44 minutes, so as to obtain the peanut milk product.

In a second aspect, the present disclosure provides a peanut milk product which is prepared by a process as described above. Such peanut milk product can alleviate at least one of the drawbacks of the prior art.

In a third aspect, the present disclosure provides a method for improving gut health, which can alleviate at least one of the drawbacks of the prior art, and which includes administering to a subject in need thereof the aforesaid peanut milk product.

In a fourth aspect, the present disclosure provides a canned food, which can alleviate at least one of the drawbacks of the prior art, and which includes the aforesaid peanut milk product.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a process for producing a peanut milk product, which includes:
(a) subjecting a milk material to a hydrolysis reaction with lactase and transglucosidase at a temperature ranging from 40° C. to 60° C. for a time period ranging from 50 minutes to 70 minutes, so as to obtain a first milk product containing galacto-oligosaccharides (GOS) and isomalto-oligosaccharides (IMO), the first milk product being optionally further subjected to heating at a temperature ranging from 70° C. to 100° C. to inactivate the lactase and transglucosidase, and subsequently to dilution with water, so as to obtain a second milk product;
(b) immersing peanut kernels in water at a temperature ranging from 70° C. to 80° C. for a time period ranging from 3 hours to 4 hours, so as to obtain swelled peanut kernels;
(c) subjecting the swelled peanut kernels obtained in step (b) to a steam cooking treatment at a temperature ranging from 105° C. to 125° C. for a time period ranging from 5 minutes to 30 minutes, so as to obtain steamed peanut kernels; and
(d) admixing the first milk product or the second milk product obtained in step (a) with the steamed peanut kernels obtained in step (c), followed by subjecting a mixture thus obtained to a heating treatment at a temperature ranging from 120° C. to 130° C. for a time period ranging from 36 minutes to 44 minutes, so as to obtain the peanut milk product.

According to the present disclosure, in step (a), the milk material may contain milk solids. Examples of the milk material may include, but are not limited to, cow's milk, goat's milk, and sheep's milk. In certain embodiments, the milk material is prepared by admixing milk powder with water.

According to the present disclosure, in step (a), the milk material may contain 30 wt % to 50 wt % of milk solids, the lactase may be present in an amount ranging from 0.01 wt % to 0.5 wt %, and the transglucosidase may be present in an amount ranging from 0.05 wt % to 0.6 wt %, based on the total weight of the milk material. By virtue of such amounts, the first milk product may have the desired contents of galacto-oligosaccharides and isomalto-oligosaccharides, and it is beneficial to control the time of the hydrolysis reaction to prevent spoilage of the first milk product, thereby making the production cost of the first milk product in line with economic benefits.

In certain embodiments, to enhance the aroma of the dairy product, in step (a), the milk material may contain 40 wt % of milk solids, the lactase may be present in an amount ranging from 0.04 wt % to 0.32 wt %, and the transglucosidase may be present in an amount ranging from 0.1 wt % to 0.5 wt %, based on the total weight of the milk material.

In an exemplary embodiment, the milk material may contain 40 wt % of milk solids, the lactase may be present in an amount of 0.1 wt %, and the transglucosidase may be present in an amount ranging from 0.25 wt % to 0.35 wt %, based on the total weight of the milk material.

In an exemplary embodiment, in order to make the hydrolysis reaction has a better reaction rate and be economical, and make the milk product has a high content of galacto-oligosaccharides and isomalto-oligosaccharides, in step (a), the hydrolysis reaction is conducted at 50° C. for 60 minutes.

According to the present disclosure, in step (a), lactose in the milk material may be hydrolyzed by lactase. Therefore, the first milk product thus obtained contains no lactose, and contains milk solids, lactase, transglucosidase, galacto-oligosaccharides, and isomalto-oligosaccharides.

In certain embodiments, in step (a), the first milk product may contain 5 wt % to 7 wt % of galacto-oligosaccharides and 3 wt % to 4 wt % of isomalto-oligosaccharides, based on the total weight of the first milk product.

According to the present disclosure, in step (a), after the first milk product is further subjected to the heating at a temperature ranging from 70° C. to 100° C. to inactivate the lactase and transglucosidase, an enzyme-inactivated milk product is obtained. In certain embodiments, the heating for enzyme inactivation may be conducted at a temperature ranging from 85° C. to 90° C. In an exemplary embodiment, the heating for enzyme inactivation is conducted at 90° C.

According to the present disclosure, after the enzyme-inactivated milk product is further diluted with water, the second milk product, which has a similar flavor and texture to cow's milk, is obtained.

In certain embodiments, the second milk product may contain 10 wt % to 15 wt % of milk solids, 1.25 wt % to 2.625 wt % of galacto-oligosaccharides, and 0.75 wt % to 1.5 wt % of isomalto-oligosaccharides, based on the total weight of the second milk product.

In an exemplary embodiment, the second milk product may contain 12 wt's of milk solids, 1.5 wt % to 2.1 wt % of galacto-oligosaccharides, and 0.9 wt % to 1.2 wt % of isomalto-oligosaccharides, based on the total weight of the second milk product.

In an exemplary embodiment, in step (d), the steamed peanut kernels obtained in step (c) are admixed with the first milk product obtained in step (a). In another exemplary embodiment, in step (d), the steamed peanut kernels obtained in step (c) are admixed with the second milk product obtained in step (a).

According to the present disclosure, the peanut kernels used in step (b) are the peanut seeds without seed coats. The appearance of the peanut kernels has no coking and discoloration. In addition, the peanut kernels have no musty smell, no yellow koji smell, no insect body, no moth-eaten decay, or no corruption, and are not admixed with other impurities.

The origin of the peanut seeds may include, but is not limited to, the United States, Taiwan, China, Nigeria, and India.

The varieties of peanut seeds may include, but are not limited to, Tainan No. 11 peanuts, Tainan No. 9 peanuts, Tainan No. 12 peanuts, red skin peanuts, China's peanuts, Tainan NO. 17 peanuts, white skin peanuts, and Hualien No. 1 peanuts.

According to the present disclosure, in step (c), the swelled peanut kernels obtained in step (b) are subjected to the steam cooking treatment to improve the texture of the peanut kernels, and the steamed peanut kernels thus obtained may have a rate of qualified doneness degree not lower than 90%, and the can containing the abovementioned steamed peanut kernels may have a rate of qualified filling quality not lower than 90%.

In certain embodiments, in step (c), the steam cooking treatment may be conducted at a temperature ranging from 110° C. to 120° C. for a time period ranging from 10 minutes to 25 minutes. In other embodiments, the steam cooking treatment may be conducted at a temperature ranging from 110° C. to 120° C. for a time period ranging from 14 minutes to 18 minutes. In an exemplary embodiment, the steam cooking treatment is conducted at 116° C. for 16 minutes.

Before the immersing treatment and the steam cooking treatment, the color of the peanut kernels is straw yellow, and the peanut kernels have hard and inelastic textures when pressed by hand.

The color of the swelled peanut kernels obtained in step (b) is light beige, and the swelled peanut kernels have hard and slightly elastic textures when pressed by hand. According to the RGB color model, the light beige color has the RGB values of red: 241, green: 235, and blue: 232.

The color of the steamed peanut kernels obtained in step (c) is goose yellow, and the steamed peanut kernels have a grainy taste and a peanut flavor. According to the RGB color model, the goose yellow color has the RGB values of red: 227, green: 200, and blue: 167.

By virtue of the immersing treatment and the steam cooking treatment, the texture of the peanut kernels can be improved. Therefore, in step (d), when the steamed peanut kernels obtained in step (c) are admixed with the first milk product or the second milk product obtained in step (a), the content ratio of the soup to the peanut kernels in the peanut milk product can achieve a better balance, so that the peanut milk product has a good flavor and taste.

According to the present disclosure, in step (d), the heating treatment may be sterilization, so that the peanut milk product may have a longer shelf life, and the texture and flavor of the steamed peanut kernels may be improved.

In certain embodiments, in step (d), the sterilization may be conducted at a temperature ranging from 127° C. to 128° C.

According to the present disclosure, in step (d), the first milk product or the second milk product and the steamed peanut kernels may be admixed in a closed environment, followed by conducting the heating treatment. In certain embodiments, the first milk product or the second milk product and the steamed peanut kernels may be placed in a sealed can, followed by conducting the heating treatment.

The peanut milk product prepared according to the process of the present disclosure has a good peanut flavor, and the peanut granules in the peanut milk product have good texture and mouthfeel. In addition, the peanut milk product can effectively prevent the imbalance of the gut microbiota, and hence is capable of maintaining a healthy gut microbiota and improving gut health.

Therefore, the present disclosure provides a peanut milk product, which is produced by a process described above.

According to the present disclosure, the peanut milk product contains greater than 1 wt % of galacto-oligosaccharides and greater than 0.5 wt % of isomalto-oligosaccharides, based on the total weight of the peanut milk product. In certain embodiments, the peanut milk product contains greater than 1 wt % and not greater than 2 wt % of galacto-oligosaccharides, and greater than 0.5 wt % and not greater than 1 wt % of isomalto-oligosaccharides, based on the total weight of the peanut milk product.

The present disclosure also provides a canned food including the aforesaid peanut milk product.

Moreover, the present disclosure provides a method for improving gut health, which includes administering to a subject in need thereof the aforesaid peanut milk product.

According to the present disclosure, the peanut milk product can increase intestinal probiotics (such as Bifidobacterium spp. and Lactobacillus spp.) and reduce the growth of intestinal harmful bacteria (such as Clostridium perfringens and Coliform bacteria).

The disclosure will be further described by way of the following examples. However, it should be understood that the following examples are solely intended for the purpose of illustration and should not be construed as limiting the disclosure in practice.

### EXAMPLES

### 1. Effect of Various Conditions of Immersing Treatment on Quality of Peanut Kernels

56.7 kg of peanut kernels were completely covered with water, followed by immersion under different time and temperature conditions as shown in Table 1 below.

The swelled peanut kernels thus obtained were subjected to texture evaluation as follows. 100 of the swelled peanut kernels were equally distributed to 5 tasters. The color of each of the distributed twenty swelled peanut kernels was visually observed by the respective taster, and the texture of each of the distributed twenty swelled peanut kernels was evaluated by the respective taster through pressing. If the color of the swelled peanut kernel was light beige (according to the RGB color model, the light beige color had the RGB values of red: 241, green: 235, and blue: 232), and if the swelled peanut kernel had hard and slightly elastic texture when pressed by hand, the texture of the swelled peanut kernel was considered qualified.

The qualified texture rate (%) was calculated using the following Equation (I):

A=(B/C)×100 (I)

where A=qualified texture rate (%)
B=number of swelled peanut kernels with qualified texture
C=total number of swelled peanut kernels (i.e., 100)

The results are shown in Table 1 below.

TABLE 1

| | | Temperature (° C.) | | | | | |
|---|---|---|---|---|---|---|---|
| | | 50 | 60 | 70 | 80 | 90 | 100 |
| | | Qualified texture rate (%) | | | | | |
| Time | 1 | <90 | <90 | <90 | <90 | <95 | <95 |
| (hour) | 2 | <90 | <90 | <95 | <95 | <95 | <95 |
| | 3 | <90 | <95 | >95 | >95 | <95 | <95 |
| | 4 | <95 | <95 | >95 | >95 | <95 | <95 |
| | 5 | <95 | <95 | <95 | <95 | <95 | <90 |
| | 6 | <95 | <95 | <95 | <95 | <90 | <90 |

As shown in Table 1, when the immersing treatment was conducted at a temperature ranging from 70° C. to 80° C. for a time period ranging from 3 hours to 4 hours, the swelled peanut kernels thus obtained had a qualified texture rate greater than 95%, indicating that the swelled peanut kernels prepared using such immersing treatment conditions have good texture, which is beneficial for filling the swelled peanut kernels into cans later, and which also enables peanut granules in a peanut milk product produced from the swelled peanut kernels to have good texture and mouthfeel.

Therefore, the abovementioned immersing treatment conditions were used for the following experiments.

### 2. Effect of Various Conditions of Steam Cooking Treatment on Quality of Peanut Kernels

A suitable amount of peanut kernels were subjected to an immersing treatment at a temperature ranging from 70° C. to 80° C. for a time period ranging from 3 hours to 4 hours. 75.6 kg of the resultant swelled peanut kernels were then subjected to a steam cooking treatment under different time and temperature conditions as shown in Table 2 below.

Thereafter, the steamed peanut kernels obtained using different steam cooking treatment conditions were subjected to doneness assessment as follows. 100 steamed peanut kernels were equally distributed to 5 tasters, and the twenty distributed steamed peanut kernels were tasted by the respective taster.

The doneness degrees of the twenty steamed peanut kernels were assessed by the respective taster. The doneness degree was rated into the following categories: “over-cooked” indicating that the steamed peanut kernel could be crushed by tongue or had a creamy texture; “half-cooked to fully-cooked” indicating that the steamed peanut kernel had a grainy taste and couldn't be crushed by tongue; and “undercooked to less than half-cooked” indicating that the steamed peanut kernel had a hard, inelastic and crisp taste, and couldn't be crushed by tongue. When the steamed peanut kernel was rated half-cooked to fully-cooked, its doneness degree was assessed as qualified.

The rate of qualified doneness degree (%) was calculated using the following Equation (II):

D=(E/F)×100 (II)

where D=rate of qualified doneness degree (%)
E=number of steamed peanut kernels with qualified doneness degree
F=total number of steamed peanut kernels (i.e., 100)

In addition, a plurality of cans were respectively filled with the steamed peanut kernels obtained using different steam cooking treatment conditions, so as to obtain a plurality of cans containing the steamed peanut kernels. Subsequently, the cans were subjected to random sampling, and the weight of the steamed peanut kernels in the respective sample can was measured. When the weight of the steamed peanut kernels fell within the range of the predetermined filling weight ±8% of the weight error, the filling quality of the sample can was assessed as qualified.

The rate of qualified filling quality (%) was calculated using the following Equation (III):

G=(H/I)×100 (III)

where G=rate of qualified filling quality (%)
H=number of sample cans with qualified filling quality
I=total number of sample cans

The results are shown in Table 2 below.

TABLE 2

| | Temperature (° C.) | | | | | | |
|---|---|---|---|---|---|---|---|
| | 100 | 110 | 110 | 116 | 120 | 120 | 130 |
| Time | 16 | 16 | 25 | 16 | 10 | 16 | 16 |
| (minute) | | | | | | | |
| Rate of | 88 | 90 | 92 | 92 | 92 | 92 | 84 |
| qualified | | | | | | | |
| doneness | | | | | | | |
| degree (%) | | | | | | | |
| Rate of | <90 | 91.6 | 92.1 | 93.4 | 91.4 | 92.8 | <90 |
| qualified | | | | | | | |
| filling | | | | | | | |
| quality (%) | | | | | | | |

As shown in Table 2, when the steam cooking treatment was conducted at a temperature ranging from 110° C. to 120° C. for a time period ranging from 10 minutes to 25 minutes, the steamed peanut kernels thus obtained had a rate of qualified doneness degree not lower than 90%, and the sample cans containing the abovementioned steamed peanut kernels had a rate of qualified filling quality not lower than 90%.

In particular, when the steam cooking treatment was conducted at 116° C. for 16 minutes, the steamed peanut kernels thus obtained had a rate of qualified doneness degree of 92%, and the sample cans containing the abovementioned steamed peanut kernels had a rate of qualified filling quality of 93.4%. Therefore, the abovementioned steam cooking treatment conditions were used for the following experiments.

### 3. Preparation of Examples 1 to 4 and Comparative Examples 1 to 2

A peanut milk product of example 1 was prepared as follows.

In step (a), 72 kg of water and 48 kg of cow milk powder were mixed homogeneously, so as to obtain a milk material containing 40 wt % of milk solids. The milk material was then mixed with 0.1 wt % of lactase and 0.05 wt % of transglucosidase, and the resultant mixture was allowed to undergo a hydrolysis reaction at 50° C. for 60 minutes, so as to obtain a first milk product. Thereafter, the first milk product was subjected to heating at 90° C. to inactivate the lactase and the transglucosidase, followed by mixing with 280 kg of water, so as to obtain a second milk product containing galacto-oligosaccharides (GOS) and isomalto-oligosaccharides (IMO).

In step (b), 56.7 kg of peanut kernels were immersed in water at 75° C., followed by stirring for 3.5 hours to obtain swelled peanut kernels.

In step (c), the swelled peanut kernels obtained in step (b) were subjected to a steam cooking treatment at 116° C. for 16 minutes, so as to obtain steamed peanut kernels. A can was filled with 60 g of the steamed peanut kernels thus obtained using a filling machine.

In step (d), the can containing the steamed peanut kernels was further filled with the second milk product obtained in step (a) using the filling machine, and the level height of the second milk product was brought to ¾ of the height of the can. The can containing the steamed peanut kernels and the second milk product was then subjected to a degassing treatment in a degassing box (82±3° C.) Thereafter, the degassed can was further filled with the second milk product, so that the total weight of the steamed peanut kernels and the second milk product in the degassed can was 340 g. Subsequently, the can was sealed, followed by sterilization in a horizontal autoclave (128° C.) for 40 minutes, so as to obtain a canned food containing a peanut milk product.

The preparation procedures for the peanut milk product of each of examples 2 to 4 and comparative examples 1 to 2 were similar to that of the peanut milk product of example 1, except that: in step (a), different amounts of lactase and transglucosidase were used for the hydrolysis reaction.

The milk solids content of the milk material in each of examples 1 to 4 and comparative examples 1 to 2, and the operation conditions for making the peanut milk products of examples 1 to 4 and comparative examples 1 to 2 are summarized in Table 3 below.

TABLE 3

| | | | | | | Comparative | |
|---|---|---|---|---|---|---|---|
| | | Example | | | | example | |
| | | 1 | 2 | 3 | 4 | 1 | 2 |
| Milk solids content of | | 40 | 40 | 40 | 40 | 40 | 40 |
| milk material (wt %) | | | | | | | |
| Lactase (wt %) | | 0.1 | 0.1 | 0.1 | 0.1 | — | 0.1 |
| Transglucosidase (wt %) | | 0.05 | 0.1 | 0.2 | 0.3 | — | — |
| Hydrolysis | Temperature | 50 | 50 | 50 | 50 | 50 | 50 |
| | (° C.) | | | | | | |
| reaction | Time | 60 | 60 | 60 | 60 | 60 | 60 |
| | (minute) | | | | | | |
| Temperature for enzyme | | 90 | 90 | 90 | 90 | 90 | 90 |
| inactivation (° C.) | | | | | | | |
| Immersing | Temperature | 75 | 75 | 75 | 75 | 75 | 75 |
| treatment | (° C.) | | | | | | |
| | Time (hour) | 3.5 | 3.5 | 3.5 | 3.5 | 3.5 | 3.5 |
| Steam cooking | Temperature | 116 | 116 | 116 | 116 | 116 | 116 |
| treatment | (° C.) | | | | | | |
| | Time | 16 | 16 | 16 | 16 | 16 | 16 |
| | (minute) | | | | | | |
| Sterilization | Temperature | 128 | 128 | 128 | 128 | 128 | 128 |
| | (° C.) | | | | | | |
| | Time | 40 | 40 | 40 | 40 | 40 | 40 |
| | (minute) | | | | | | |

### 4. Quality Evaluation of Examples 1 to 4 and Comparative Examples 1 to 2

### Methods:

### A. Determination of IMO Content

The peanut milk product, first milk product, and second milk product of the respective one of examples 1 to 4 and comparative examples 1 to 2 obtained in Section 3 were used as test samples and were subjected to the following analyses.

A respective one of the test samples was subjected to a homogenization treatment. The resultant homogenized test sample was quantified with deionized water, followed by filtration using a filter membrane having a pore size of 0.45 μm, so as to obtain a test solution, and then high performance liquid chromatography (HPLC) analysis was conducted, so as to determine the IMO content therein.

HPLC analysis was performed using an HPLC system (1260 Infinity, Agilent Technologies, Inc.) equipped with a RI detector. The column and operation conditions for HPLC are as follows: Hypersil-NH2 column (length: 250 mm; inner diameter: 4.6 mm; particle size: 5 μm); mobile phase: 75% acetonitrile; and flow rate: 1.0 mL/min.

### B. Determination of GOS Content

The peanut milk product, first milk product, and second milk product of the respective one of examples 1 to 4 and comparative examples 1 to 2 obtained in Section 3 were used as test samples and were subjected to the following analyses.

A respective one of the test samples was subjected to a homogenization treatment, and the resultant homogenized test sample was then mixed with a suitable amount of a 10% glycerin solution (serving as an internal standard), followed by adding a suitable amount of a 20% salicylic acid solution. The resultant mixture was quantified with deionized water and was mixed evenly, followed by centrifugation to obtain a supernatant. The supernatant was then subjected to filtration using a filter membrane having a pore size of 0.45 μm, so as to obtain a test solution.

A portion of the respective one of the test solutions was subjected to HPLC analysis using an HPLC system (1260 Infinity, Agilent Technologies, Inc.) equipped with a RI detector, so as to determine the total content of GOS and lactose therein. The column and operation conditions for HPLC are as follows: Shodex Sugar KS-802 column (length: 300 mm; inner diameter: 8 mm; particle size: 6 μm); mobile phase: deionized water; flow rate: 0.5 mL/min; and column temperature: 80° C.

In addition, another portion of the respective one of the test solutions was subjected to HPLC analysis using an HPLC system (1260 Infinity, Agilent Technologies, Inc.) equipped with a RI detector, so as to determine the lactose content therein. The column and operation conditions for HPLC are as follows: COSMOSIL Sugar-D (length: 250 mm; inner diameter: 4.6 mm; particle size: 5 μm); mobile phase: 70% acetonitrile; and flow rate: 1.0 mL/min.

The GOS content was calculated using the following Equation (IV):

J=K−L (IV)

where J=GOS content

K=total content of GOS and lactose

L=lactose content

### C. Evaluation of Peanut Flavor

The peanut milk product of each of examples 1 to 4 and comparative examples 1 to 2 obtained in Section 3 was tasted by 43 tasters, and each peanut milk product was rated for its peanut flavor. Each taster rinsed his mouth before and after the tasting, so as to provide peanut flavor scores without interference from other food flavors.

The peanut flavor was recorded by the tasters and quantified by scoring on a scale from 1 to 5. The higher scale indicated the least peanut flavor (i.e. scale 1 indicated the strongest peanut flavor in the viewpoint of the tasters, and scale 5 indicated the weakest peanut flavor in the viewpoint of the tasters). The number of tasters corresponding to each score was counted, and the total peanut flavor score of the respective peanut milk product was then calculated.

### Results:

The results of the quality evaluation are shown in Table 4. It can be seen from Table 4 that the total peanut flavor scores of the peanut milk products of examples 1 to 4 were lower than those of the peanut milk products of comparative examples and 2 (the lower total peanut flavor score indicated the stronger peanut flavor).

These results indicate that using lactase and transglucosidase to hydrolyze the milk material can obtain the first milk product containing GOS and IMO, and the peanut milk product prepared therefrom can have good peanut flavor.

TABLE 4

| | | | | | | Comparative | |
|---|---|---|---|---|---|---|---|
| | | Example | | | | example | |
| | | 1 | 2 | 3 | 4 | 1 | 2 |
| First milk | GOS | 5.64 | 5.77 | 6.10 | 6.76 | 0 | 5.16 |
| product | content | | | | | | |
| | IMO | 2.22 | 2.41 | 2.38 | 3.16 | 0 | 0 |
| | content | | | | | | |
| Second | GOS | 1.69 | 1.73 | 1.83 | 2.03 | 0 | 1.55 |
| milk | content | | | | | | |
| product | (wt %) | | | | | | |
| | IMO | 0.67 | 0.72 | 0.71 | 0.95 | 0 | 0 |
| | content | | | | | | |
| | (wt %) | | | | | | |
| Peanut | GOS | 1.39 | 1.42 | 1.51 | 1.67 | 0 | 1.28 |
| milk | content | | | | | | |
| product | (wt %) | | | | | | |
| | IMO | 0.55 | 0.59 | 0.59 | 0.78 | 0 | 0 |
| | content | | | | | | |
| | (wt %) | | | | | | |
| Peanut | Scale 1 | 0* | 1* | 2* | 40* | 0* | 0* |
| flavor of | Scale 2 | 8* | 16* | 17* | 2* | 0* | 0* |
| peanut | Scale 3 | 7* | 17* | 18* | 0* | 0* | 1* |
| milk | Scale 4 | 26* | 8* | 6* | 1* | 2* | 2* |
| product | Scale 5 | 2* | 1* | 0* | 0* | 41* | 40* |
| | Total | 151 | 121 | 114 | 48 | 213 | 211 |
| | scale | | | | | | |

*The number of tasters giving the score as indicated.

### 5. Preparation of Example 5 and Comparative Examples 3 to 8

The preparation procedures for the peanut milk product of each of example 5 and comparative examples 3 to 8 were similar to that of the peanut milk product of example 4, except that: in step (d), sterilization was performed under different conditions of temperature and time.

The milk solids content of the milk material in each of example 5 and comparative examples 3 to 8, and the operation conditions for making the peanut milk products of example 5 and comparative examples 3 to 8 are summarized in Table 5 below.

TABLE 5

| | | Example | Comparative example | | | | | |
|---|---|---|---|---|---|---|---|---|
| | | 5 | 3 | 4 | 5 | 6 | 7 | 8 |
| Milk solids content of | | 40 | 40 | 40 | 40 | 40 | 40 | 40 |
| milk material (wt %) | | | | | | | | |
| Lactase (wt %) | | 0.1 | 0.1 | 0.1 | 0.1 | 0.1 | 0.1 | 0.1 |
| Transglucosidase (wt %) | | 0.3 | 0.3 | 0.3 | 0.3 | 0.3 | 0.3 | 0.3 |
| Hydrolysis | Temperature | 50 | 50 | 50 | 50 | 50 | 50 | 50 |
| reaction | (° C.) | | | | | | | |
| | Time | 60 | 60 | 60 | 60 | 60 | 60 | 60 |
| | (minute) | | | | | | | |
| Temperature for enzyme | | 90 | 90 | 90 | 90 | 90 | 90 | 90 |
| inactivation (° C.) | | | | | | | | |
| Immersing | Temperature | 75 | 75 | 75 | 75 | 75 | 75 | 75 |
| treatment | (° C.) | | | | | | | |
| | Time (hour) | 3.5 | 3.5 | 3.5 | 3.5 | 3.5 | 3.5 | 3.5 |
| Steam | Temperature | 116 | 116 | 116 | 116 | 116 | 116 | 116 |
| cooking | (° C.) | | | | | | | |
| treatment | Time | 16 | 16 | 16 | 16 | 16 | 16 | 16 |
| | (minute) | | | | | | | |
| Sterilization | Temperature | 127 | 127 | 127 | 127 | 127 | 100 | 110 |
| | (° C.) | | | | | | | |
| | Time | 40 | 30 | 35 | 45 | 50 | 40 | 40 |
| | (minute) | | | | | | | |

### 6. Quality Evaluation of Examples 4 to 5 and Comparative Examples 3 to 8

### Methods:

### A. Evaluation of Integrity of Peanut Granules

The integrity of peanut granules of the peanut milk product of each of examples 4 to 5 and comparative examples 3 to 8 Was evaluated by 46 tasters.

The integrity of peanut granules was recorded by the tasters and quantified by scoring on a scale from to 9. The higher scale indicated the higher integrity of the peanut granules (i.e. scale indicated the worst peanut granule integrity in the viewpoint of the tasters, and scale 9 indicated the best peanut granule integrity in the viewpoint of the tasters). The experimental data are expressed as mean.

### B. Evaluation of Peanut Flavor

The peanut milk product of each of examples 4 to 5 and comparative examples 3 to 8 was tasted by 46 tasters, and each peanut milk product was rated for its peanut flavor. Each taster rinsed his mouth before and after the tasting, so as to provide peanut flavor scores without interference from other food flavors.

The peanut flavor was recorded by the tasters and quantified by scoring on a scale from 1 to 9. The higher scale indicated the stronger peanut flavor (i.e. scale 1 indicated the weakest peanut flavor in the viewpoint of the tasters, and scale 9 indicated the strongest peanut flavor in the viewpoint of the tasters). The experimental data are expressed as mean.

### C. Evaluation of Texture Characteristics of Peanut Granules

The peanut milk product of each of examples 4 to 5 and comparative examples 3 to 8 was tasted by 46 tasters, and each peanut milk product was rated for its texture characteristics. Each taster rinsed his mouth before and after the tasting, so as to provide texture scores without interference from other foods.

The texture characteristics, including creamy texture and texture homogeneity of peanut granules, were recorded by the tasters and quantified by scoring on a scale from 1 to 9. The higher scale indicated the higher texture quality (i.e. scale 1 indicated the least creamy texture and worst texture homogeneity in the viewpoint of the tasters, and scale 9 indicated the most creamy texture and the best texture homogeneity in the viewpoint of the tasters). The experimental data are expressed as mean.

### D. Determination of Hardness of Peanut Granules

The peanut milk product of each of example 4 and comparative examples 7 to 8 and a commercially available peanut milk product were subjected to determination of hardness of peanut granules using a TA.XT2i Texture analyzer (TA Instruments) and the operating conditions shown in Table 6 below.

TABLE 6

| Probe | P/6 mm | |
|---|---|---|
| Pre-test Speed | 1 | mm/sec |
| Test Speed | 1 | mm/sec |
| Post-test Speed | 5 | mm/sec |
| Target Mode | Strain | |
| Strain | 75% | |
| Time between two compressions | 5 | sec |
| Trigger Type | Auto (Force) | |
| Trigger Force | 5 | g |

15 peanut granules were selected from each peanut milk product for testing, and the experimental data are expressed as mean±standard deviation (SD). When the average hardness of peanut granules was less than 135 g, the average hardness of peanut granules was considered qualified. When the standard deviation of the hardness was less than 20 g, the uniformity of hardness of peanut granules was considered qualified.

In addition, the maximum hardness of the peanut granules was defined as the upper limit value of hardness, and the minimum hardness of the peanut granules was defined as the lower limit value of hardness.

### Results:

The results of the quality evaluation are shown in Tables 7 and 8. It can be seen from Table 7 that the scores of peanut flavor, integrity of peanut granules, and texture characteristics of peanut granules for the peanut milk products of examples 4 to 5 were higher than those for the peanut milk products of comparative examples 3 to 8.

These results indicate that when the sterilization is conducted at 127° C. or 128° C. for 40 minutes, the resultant peanut milk product has good integrity of peanut granules, peanut flavor, and texture characteristics of peanut granules.

In addition, it can be seen from Table 8 that the average hardness of the peanut granules in the peanut milk product of example 4 was less than 135 g, and the standard deviation of the hardness was less than 20 g.

These results indicate that when the sterilization is conducted at 128° C. for 40 minutes, the peanut granules of the peanut milk product have excellent hardness and uniformity of hardness, and hence have a good mouthfeel.

TABLE 7

| | Example | | Comparative example | | | | | |
|---|---|---|---|---|---|---|---|---|
| | 4 | 5 | 3 | 4 | 5 | 6 | 7 | 8 |
| Peanut | 6.2 | 6.6 | 4.6 | 4.8 | 4.9 | 4.9 | 4.55 | 4.4 |
| flavor | | | | | | | | |
| Integrity | 6.9 | 7.1 | 7.0 | 7.0 | 6.2 | 6.2 | 6.95 | 6.85 |
| of peanut | | | | | | | | |
| granules | | | | | | | | |
| Creamy | 7.5 | 6.1 | 5.1 | 5.0 | 5.7 | 4.9 | 2.05 | 3.95 |
| texture of | | | | | | | | |
| peanut | | | | | | | | |
| granules | | | | | | | | |
| Texture | 7.6 | 7.0 | 6.2 | 6.2 | 6.5 | 5.0 | 3.95 | 4.15 |
| homogeneity | | | | | | | | |
| of peanut | | | | | | | | |
| granules | | | | | | | | |

TABLE 8

| | | | | Commercially |
|---|---|---|---|---|
| | | | | available |
| | Example | Comparative example | | peanut milk |
| | 4 | 7 | 8 | product |
| Upper limit | 130.69 | 706.64 | 296.66 | 245.14 |
| value of | | | | |
| hardness | | | | |
| (g) | | | | |
| Lower limit | 95.41 | 320.92 | 191.30 | 157.00 |
| value of | | | | |
| hardness | | | | |
| (g) | | | | |
| Average | 113.05 ± 17.64 | 513.78 ± 192.86 | 243.98 ± 52.68 | 201.07 ± 44.07 |
| hardness | | | | |
| (g) | | | | |
| (mean ± SD) | | | | |

### 7. Evaluation for the Effect of Peanut Milk Product According to This Disclosure on Restoring Healthy Gut Flora

### Materials and Methods:

### A. Test Animals

Sprague Dawley (S.D.) rats (8 weeks old, with a body weight of approximately 230 g) were purchased from BioLASCO Taiwan Co., Ltd. The S.D. rats were kept in an animal room with an independent air conditioning system under the following laboratory conditions: a temperature of 22±3° C. and a relative humidity of 55±15%. Furthermore, water and feed were provided ad libitum for all experimental animals.

B. Feeding with Peanut Milk Product of Present Disclosure

The S.D. rats were divided into 2 groups, including one experimental group and one comparative group. The S.D. rats of the experimental group were fed with the peanut milk product of example 1, and the S.D. rats of the comparative group were fed with reverse osmosis (RO) water. Each rat was fed for a total period of 4 weeks.

Prior to the feeding of the peanut milk product (i.e., at Week 0) and at the end of Week 4 after starting the feeding of the peanut milk product, a fecal sample was obtained from each rat, and was subjected to determination of the numbers of Bifidobacterium spp., Lactobacillus spp., Clostridium perfringens, and Escherichia coli (E. coli) using standard plate count. For standard plate count, Bifidobacterium iodoacetate medium 25 (BIM-25) was used to determine Bifidobacterium spp., MRS (De Man, Rogosa and Sharpe) medium was used to determine Lactobacillus spp., tryptose sulfite cycloserine (TSC) agar was used to determine Clostridium perfringens, and chromogenic E. coli/coliform agar was used to determine E. coli.

### Results:

As shown in Table 9 below, regarding the result of the experimental group, the numbers of Eifidobacterium spp. and Lactobacillus spp. (i.e., probiotics) determined at the end of Week 4 were higher than those determined at Week 0, while no significant difference was observed on the numbers of Clostridium perfringens and E. coli (i.e., pathogenic bacteria). On the contrary, regarding the result of the comparative group, no significant difference was observed on the numbers of the probiotics and pathogenic bacteria before and after the 4-week feeding period.

These results indicate that the peanut milk product of the present disclosure can increase intestinal probiotics and reduce the growth of intestinal harmful bacteria, and hence is capable of maintaining a healthy gut microbiota and improving gut health.

TABLE 9

| | |
|---|---|
| Experimental | Comparative |
| group | group |

Number of bacteria
(log₁₀CFU/g) (mean ± SD)

| | | | |
|---|---|---|---|
| Bifidobacterium | Week 0 | 6.00 ± 0.27 | 5.98 ± 0.25 |
| spp. | Week 4 | 6.26 ± 0.18^{a} | 6.00 ± 0.21 |
| Lactobacillus | Week 0 | 8.09 ± 0.14 | 8.12 ± 0.29 |
| spp. | Week 4 | 8.55 ± 0.13^{a} | 8.18 ± 0.18 |
| Clostridium | Week 0 | 6.48 ± 0.37 | 6.38 ± 0.38 |
| perfringens | Week 4 | 6.38 ± 0.49 | 6.39 ± 0.43 |
| E. coli | Week 0 | 6.27 ± 0.67 | 6.14 ± 0.14 |
| | Week 4 | 6.48 ± 0.41 | 6.31 ± 0.28 |

^{a}When the experimental data obtained at the end of Week 4 was compared to that obtained at Week 0, p < 0.05.

Summarizing the above test results, it is clear that the peanut milk product prepared according to the process of the present disclosure has good peanut flavor, and the peanut granules in the peanut milk product have good texture and mouthfeel. Moreover, by virtue of the immersing treatment and the steam cooking treatment, the sample cans containing the steamed peanut kernels have a rate of qualified filling quality not lower than 90%. In addition, the peanut milk product can effectively prevent the imbalance of the gut microbiota, and hence is capable of maintaining a healthy gut microbiota and improving gut health.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A process for producing a peanut milk product, comprising:
(a) subjecting a milk material to a hydrolysis reaction with lactase and transglucosidase at a temperature ranging from 40° C. to 60° C. for a time period ranging from 50 minutes to 70 minutes, so as to obtain a first milk product containing galacto-oligosaccharides and isomalto-oligosaccharides, the first milk product being optionally further subjected to heating at a temperature ranging from 70° C. to 100° C. to inactivate the lactase and transglucosidase, and subsequently to dilution with water, so as to obtain a second milk product;
(b) immersing peanut kernels in water at a temperature ranging from 70° C. to 80° C. for a time period ranging from 3 hours to 4 hours, so as to obtain swelled peanut kernels;
(c) subjecting the swelled peanut kernels obtained in step (b) to a steam cooking treatment at a temperature ranging from 105° C. to 125° C. for a time period ranging from 5 minutes to 30 minutes, so as to obtain steamed peanut kernels; and
(d) admixing the first milk product or the second milk product obtained in step (a) with the steamed peanut kernels obtained in step (c), followed by subjecting a mixture thus obtained to a heating treatment at a temperature ranging from 120° C. to 130° C. for a time period ranging from 36 minutes to 44 minutes, so as to obtain the peanut milk product that has superior peanut taste and aroma and has ability to improve gut microbiota.

2. The process according to claim 1, wherein in step (a), the milk material contains 30 wt % to 50 wt % of milk solids, the lactase is present in an amount ranging from 0.01 wt % to 0.5 wt %, and the transglucosidase is present in an amount ranging from 0.05 wt % to 0.6 wt %, based on the total weight of the milk material.

3. The process according to claim 1, wherein in step (a) , the first milk product contains 5 wt % to 7 wt % of galacto-oligosaccharides and 3 wt % to 4 wt % of isomalto-oligosaccharides, based on the total weight of the first milk product.

4. The process according to claim 1, wherein in step (d), the steamed peanut kernels obtained in step (c) are admixed with the first milk product obtained in step (a).

5. The process according to claim 1, wherein in step (d), the steamed peanut kernels obtained in step (c) are admixed with the second milk product obtained in step (a).

6. The process according to claim 1, wherein in step (a), the second milk product contains 10 wt % to 15 wt % of milk solids, 1.25 wt % to 2.625 wt % of galacto-oligosaccharides, and 0.75 wt % to 1.5 wt % of isomalto-oligosaccharides, based on the total weight of the second milk product.

7. The process according to claim 1, wherein in step (d), the first milk product or the second milk product and the steamed peanut kernels are admixed in a closed environment, followed by conducting the heating treatment.

8. The process according to claim 1, wherein in step (c), the steam cooking treatment was conducted at a temperature ranging from 110° C. to 120° C. for a time period ranging from 10 minutes to 25 minutes.

9. A peanut milk product, which is produced by a process according to claim 1.

10. The peanut milk product according to claim 9, which contains greater than 1 wt % of galacto-oligosaccharides and greater than 0.5 wt % of isomalto-oligosaccharides, based on the total weight of the peanut milk product.

11. A method for improving gut health, comprising administering to a subject in need thereof a peanut milk product as claimed in claim 9.

12. A canned food comprising a peanut milk product as claimed in claim 9.

13. The canned food according to claim 12, wherein the peanut milk product contains greater than 1 wt % of galacto-oligosaccharides and greater than 0.5 wt % of isomalto-oligosaccharides, based on the total weight of the peanut milk product.
